(19) United States
(12) Patent Application Publication
(10) Pub. No.: US 2008/0097530 A1
(43) Pub. Date: Apr. 24, 2008

(54) SYSTEM FOR TISSUE STIMULATION AND REGENERATION

(76) Inventors: Philip MUCCIO, Columbus, OH (US); Robert G. DENNIS, Chapel Hill, NC (US); David A. WOLF, Houston, TX (US); Marc LABBE, Houston, TX (US)

Correspondence Address: LADAS & PARRY LLP, 224 SOUTH MICHIGAN AVENUE, SUITE 1600, CHICAGO, IL, 60604, US

(21) Appl. No.: 11/874,410

(22) Filed: Oct. 18, 2007

Related U.S. Application Data
(60) Provisional application No. 60/853,636, filed on Oct. 23, 2006.

(51) Int. Cl. A61N 1/34; A61N 1/00; A61N 1/36

(52) U.S. Cl. 607/3; 607/46; 607/50

(57) ABSTRACT

A system for stimulating tissue to relieve pain and repair and/or regenerate tissue comprising a garment that has an electrode, a programmable electrical stimulation device, a time varying electromagnetic field generator, and a source that provides an electrical current. Also provided is a method wherein the electrode provides the user with a stimulating current and the time varying electromagnetic field provides the user with a time varying electromagnetic field either simultaneously, alternating, and/or sequentially.

### FIELD OF THE INVENTION

This invention relates generally to systems and devices for tissue repair and/or regeneration, and pain relief, by a combination of an electrical stimulation current and a time varying electromagnetic field, and more specifically to a system comprising a garment, at least one electrode, a programmable electrical stimulation device, a time varying electromagnetic field generator, and a source of electric current.

### BACKGROUND OF THE INVENTION

Victims of tissue injury suffer varying degrees of trauma ranging from slight pain while the tissue regenerates to near total incapacitation when the injury is to the spinal cord. If the individual is incapacitated, problems can occur in areas other than the injured area such as when an individual is confined to a bed or wheelchair and atrophy of muscles occurs or the skin breaks down in what is referred to as “bed sores”. Skin breakdown or acute pain can result in a significant loss for the individual and family, and can result in loss of job, reduction of income, depression, and overall diminished quality of life. Further, extensive injury can be beyond the financial capabilities of the family. Thus, there is a need for a system, method and/or device to assist individuals with tissue repair to regenerate the tissue and minimize the pain while doing so.

### SUMMARY OF THE INVENTION

The following provides a summary of exemplary embodiments of the present invention. The summary is not intended to limit critical aspects or elements of the invention or to delineate its scope.

This invention is related to a system for transcutaneous tissue stimulation and regeneration of tissue comprising a garment having at least one electrode that may preferably be attachable to or embedded within the fabric of the garment; a programmable electrical stimulation device operatively connected to at least one electrode; a time varying electromagnetic field generator preferably in close proximity to one of the electrodes; and a source to supply the time varying electromagnetic field generator with electrical current to cause it to generate a time varying electromagnetic field. The garment of the system can be, but is not limited to, a vest, briefs, belt, shorts, brace, sling, immobilizer, and combinations thereof.

The present invention is related to effective management of acute and chronic pain; effective management of sports-related injury; rehabilitation and regeneration or tissue in orthopedic and sports injury; rehabilitation and regeneration of damaged or paralyzed muscle; and neurological damage or impairment. Additionally, weak muscles can be strengthened and regenerated and muscle problems can be overcome by release of endogenous opiates (natural pain inhibitor) and improved local blood circulation.

Furthermore, this invention is related to a method of regenerating, repairing, and/or stimulating tissue wherein the system delivers a direct stimulation current and a time varying electromagnetic field to the tissue to be stimulated and regenerated. The direct stimulation current and time varying electromagnetic field may be provided to the tissue alone, simultaneously, and/or in a synchronized alternating pattern, as desired.

This invention is also related to a therapeutic use of wearing the defined garment. The invention is also related to a prosthetic, method of relieving pain, and the regeneration and/or repair of tissue.

### BRIEF DESCRIPTION OF THE DRAWINGS

The accompanying drawings, which are incorporated into and form a part of the specification, schematically illustrate one or more exemplary embodiments of the invention and together with the general description given above and the detailed description given below serve to explain the principals of the invention, wherein:

FIG. 1 is a stylized front view of an individual wearing the device of this invention, wherein multiple embodiments of the wearable device are shown on a single human figure;

FIG. 1A shows a body part with the time varying electromagnetic field (“TVEMF”) generator configured as two sheets that generate the TVEMF;

FIG. 2 is a rear perspective view of an exemplary embodiment of the garment of this system configured as a vest;

FIG. 3 is a rear view of an exemplary embodiment with the garment of the system configured as briefs;

FIG. 4 is a side view of an alternate embodiment of the garment of the system of this invention;

FIG. 5 is a front view of an exemplary embodiment of the garment of the system of this invention configured as a belt;

FIG. 6 is a rear view of an exemplary embodiment of the garment of the system of this invention configured as a belt;

FIG. 6A is a view of the TVEMF generator of the system configured as a helix;

FIG. 7 is a top view of an exemplary embodiment of the electrode of the system;

FIG. 8 is a cross section showing the stimulation current application of this invention;

FIG. 9 is a cross section showing the stimulation current application of this invention;

FIG. 10 shows the electrode placement and stimulation current application placed on an individual's back; and

FIG. 11 shows the electrode placement and stimulation current application placed on an individual's shoulder's.

### DETAILED DESCRIPTION OF THE INVENTION

Exemplary embodiments of the invention are now described with reference to the Figures. Reference numerals are used throughout the detailed description to refer to the various elements and structures. For purposes of explanation, numerous specific details are set forth in the detailed description to facilitate a thorough understanding of this invention. It should be understood, however, that the present invention may be practiced without these specific details, and the invention should not be limited thereby. In other instances, however, well-known structures and devices are shown in block diagram form for purposes of simplifying the description.

The present invention relates to a wearable stimulation and tissue regeneration device for regenerating tissue and to minimize pain associated therewith. One general embodiment of this invention provides for a system for transcutaneous neural muscular stimulation as more fully described herein. Simply, a system may comprise a garment, a programmable electrical stimulation device operatively connected to at least one electrode associated with the fabric of the garment, a time varying electromagnetic field (“TVEMF”) generator associated with the garment, and a source operatively connected to the TVEMF generator.

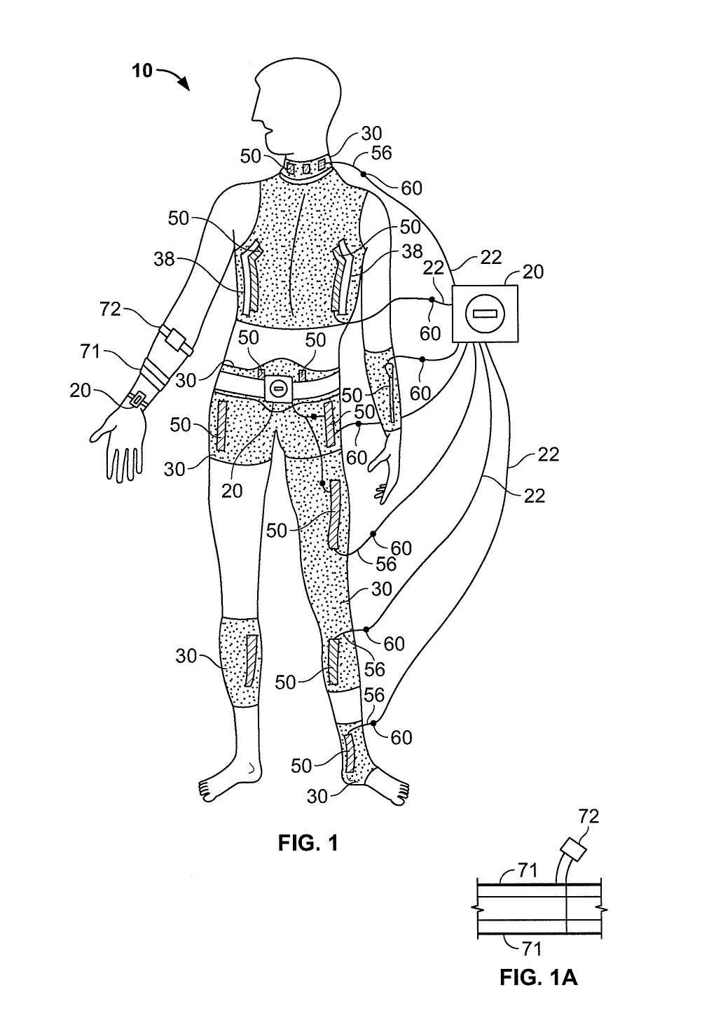

With reference now to the Figures, FIG. 1 illustrates multiple embodiments of the invention on a stylized human form. It should be realized and understood that FIG. 1 is merely illustrative in nature, as it is highly unlikely that any one person would wear all possible versions of the device at once. FIG. 1, illustrates an exemplary system 10 that includes a programmable electrical stimulation device 20 operatively connected to or otherwise in communication with one or more portions of the garment 30. The programmable electrical stimulation device 20 is operatively connected to the system 10. By the term “operatively connected,” it is intended that, in use, the source and/or the programmable electrical stimulation device can be connected by a conductive material, such as, but not limited to, a silver coated fabric and a wire, to the TVEMF generator and the electrode respectively, or alternatively may be able to remotely access the TVEMF generator and electrode respectively to control the same. The programmable electrical stimulation device 20 may be worn directly on or around the waist, the wrist, or other body part.

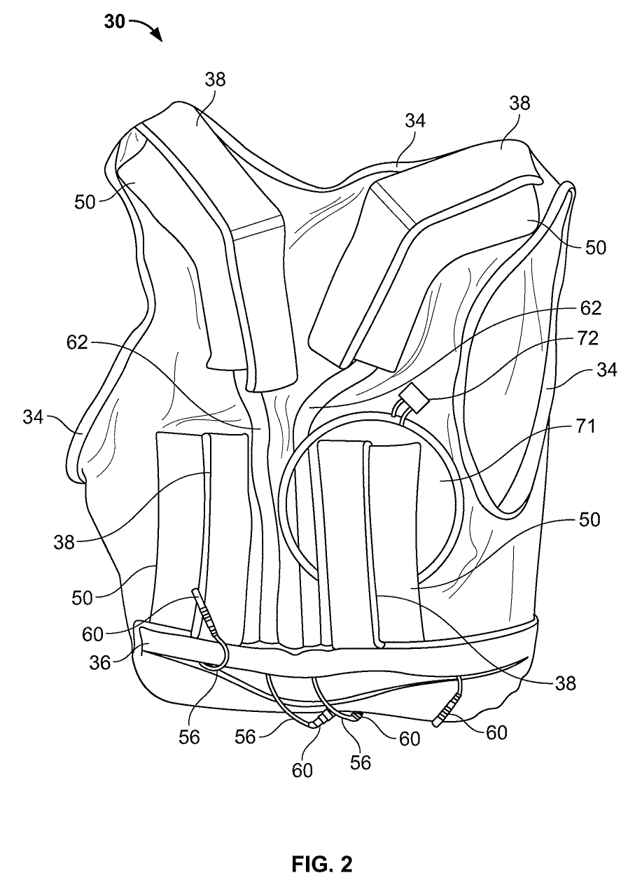

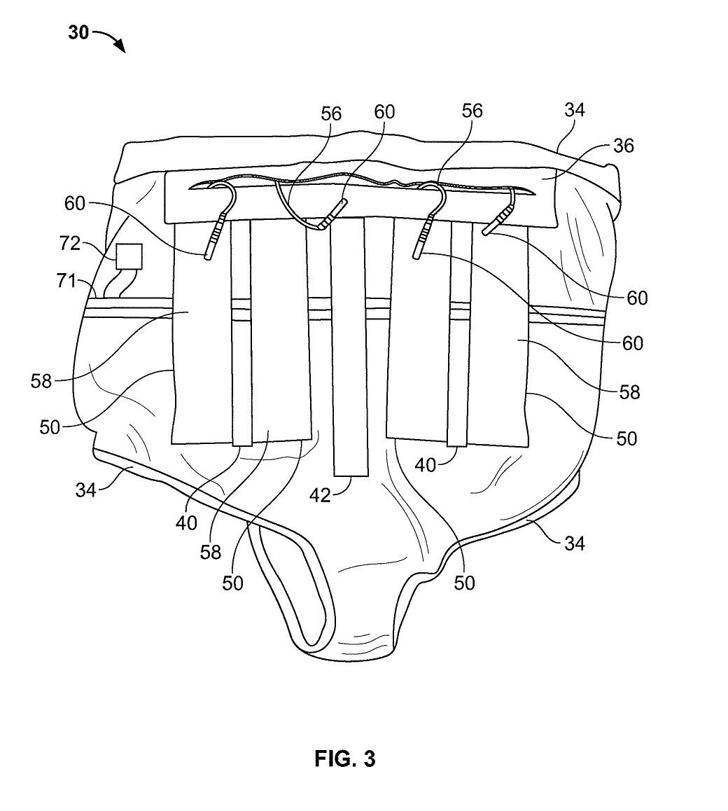

The garment 30 may preferably include multiple openings 34 (FIG. 2) and may be configured as any number of garment styles including, but not limited to, a collar, vest, sleeve, shirt, belt, shorts, briefs, trousers, sock, or a suit (see also FIGS. 2-6). Combinations of the garment 30 are also contemplated for certain applications. The garment 30 is preferably made of flexible, non-conductive fabric such as Lycra and/or spandex and may include one or more garment support members 40 and/or securing members 42 (see FIG. 3). In another embodiment, garment support members 40 function as stays or boning that help keep the garment 30 from rolling up and help maintain physical distance between the electrodes 50. In still another embodiment of the system 10, securing members 42 serve as attachment points for an “elastic wrap around” device, which may be utilized to apply additional external pressure to the electrodes 50 for the purpose of maintaining complete or nearly complete contact with the skin of the wearer of the garment. Each garment 30 also includes at least one electrode 50 that is attached to or embedded within the fabric of each garment. In operation, each electrode 50 delivers precisely controlled stimulation current to the user of the system 10.

Each electrode 50 may be situated on the top surface of the garment 30, on the bottom surface of garment 30, or may simply be sewn into the garment 30. By the term “top surface” the surface of the garment 30 that is worn on the outside, away from the body, by the user of the garment 30, is intended. By the term “bottom surface” the surface of the garment 30 that is worn on the inside, closest to the body, by the user of the garment 30, is intended. In one embodiment, a closable gel pocket or reservoir 38 may be included (see FIG. 2) with the electrode 50, particularly when the electrode 50 is situated on the top surface of garment 30.

FIG. 7 illustrates an exemplary embodiment of an individual electrode 50, which may be any number of shapes and sizes. In the embodiment illustrated in FIG. 7, the electrode 50 includes a first fabric layer 52, which may be either an absorbent material or water-barrier material. The first fabric layer 52 is also “insulating” in that it minimizes the likelihood that either the wearer/user of the garment or a person who touches the garment will be inadvertently exposed to electrical current. In this preferred embodiment of the electrode 50, an electrically conductive, silver-treated material 54 is then placed on the top surface of the first fabric layer 52. The term “silver-treated” refers to a fabric or material that has been coated, sprayed, and/or embedding with one or more layers of silver. As shown in FIG. 7, an electrical wire 56 is attached to the silver-treated material 54 for the purpose of transmitting a controlled stimulation current into and through the silver-treated material to the individual using the wearable item. As shown in the Figure, a portion of electrical wire 56 has been de-insulated and tied into a retaining knot 70. The remaining de-insulated portion 64 of electrical wire 56 and a length of the insulated portion of electrical wire 56 are attached to silver-treated material 54 by loose stitching 66 and tight stitching 68. A second fabric layer 58, which covers the silver-treated material and de-insulated portion 64, should typically be included (see FIG. 3); thus, the silver-treated material is typically sandwiched between two pieces of non-conductive fabric. In the exemplary embodiments described herein, the silver-treated material, which is commercially available in sheets or rolls may be cut and sized according to overall design of the electrode 50. The silver-treated material is typically coated with at least two layers of an organic compound or other protective substance to prolong the life of the electrode on the body.

In the exemplary embodiment, at least one, electrode 50 is operatively connected to with a programmable electrical stimulation device 20. The programmable electrical stimulation device 20 may be any of a number of devices, either off-the-shelf or custom designed and built. The programmable electrical stimulation device 20 is capable of delivering electric current to the electrodes 50 in a controllable and predictable manner. The programmable electrical stimulation device 20 may include multiple channels, may be microprocessor-controlled, may be portable, and may include a transponder for wireless operation. In one embodiment, the electrode 50 is fabricated from flexible circuitry to enable the garment to conform to the user's body, and the programmable electrical stimulation device 20 is mounted in a discrete location on the garment. The parts are modular and interchangeable as needed. The programmable electrical stimulation device 20 is powered by electric energy, for instance from a battery.

As shown in FIG. 1, which depicts multiple embodiments of the invention, electrical wires 56 are connected to lead wires 22 by connectors 60. Each connector 60 is typically a receptacle adapted to receive lead wire pins. In some embodiments of this invention, the electrodes 50 are in electrical communication with the programmable electrical stimulation device 20 by wireless or remote communication; thus, connector 60 may be or may include a transceiver or other signal-receiving device known in the art to enable remote control of or access to the electrodes 50. In this way, if the user of the system is incapacitated, or there is a concern that the user may make improper adjustments, the programmable electrical stimulation device 20 modes and parameters can be controlled remotely using a computer, the Internet, or other telecommunications devices. Further, the programmable electrical stimulation device 20 modes and parameters can be preset as either fixed, to adjust over time, be configured to react to biofeedback signals, and/or can also be designed to be smoothly adjusted by the user. Moreover, the programmable electrical stimulation device 20 can be operatively connected to sensors that detect electrical current, magnetic flux, temperature, and/or impedance, and control the same. Also, the programmable electrical stimulation device 20 can be designed to drive more than one electrode or TVEMF generator(s), which may be located at more than one location on the body, or on more than one garment, and may be operated independently or in a synchronized fashion. Alternatively, the TVEMF generator can have an independent TVEMF source that in use provides electricity to the TVEMF generator.

In some embodiments of this invention, flexible conductors 62 connect the electrode 50 to the wires 56. These flexible conductors 62 are generally flat and include one or more layers of electrically conductive silver-treated material, which is the same as, or similar to the silver-treated material included in the electrodes 50. Inclusion of one or more flexible conductors 62 within or on the garment 30 reduces the number of electrical wires 56, resulting in a more comfortable and less cumbersome garment. Also, reducing the number of electrical connections that the user must make before the system can be operated, simplifies the use of the system in general. Additionally, certain embodiments of the garment 30 include closeable pockets 36, closeble by fasteners well known in the art including, but not limited to, zippers, hook and loop closures, and buttons, and wherein the closeable pockets 36 are useful for storing/enclosing wires 56 and connectors 60 so that these items are kept out of the way of the system 10. In an embodiment, the programmable electrical stimulus device generally utilized micro-power design technology to enable battery power and portability.

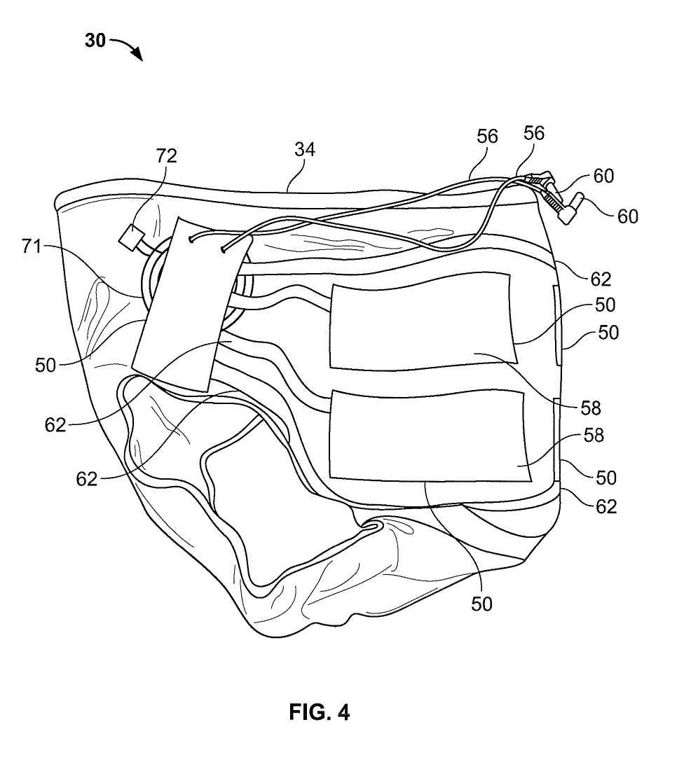

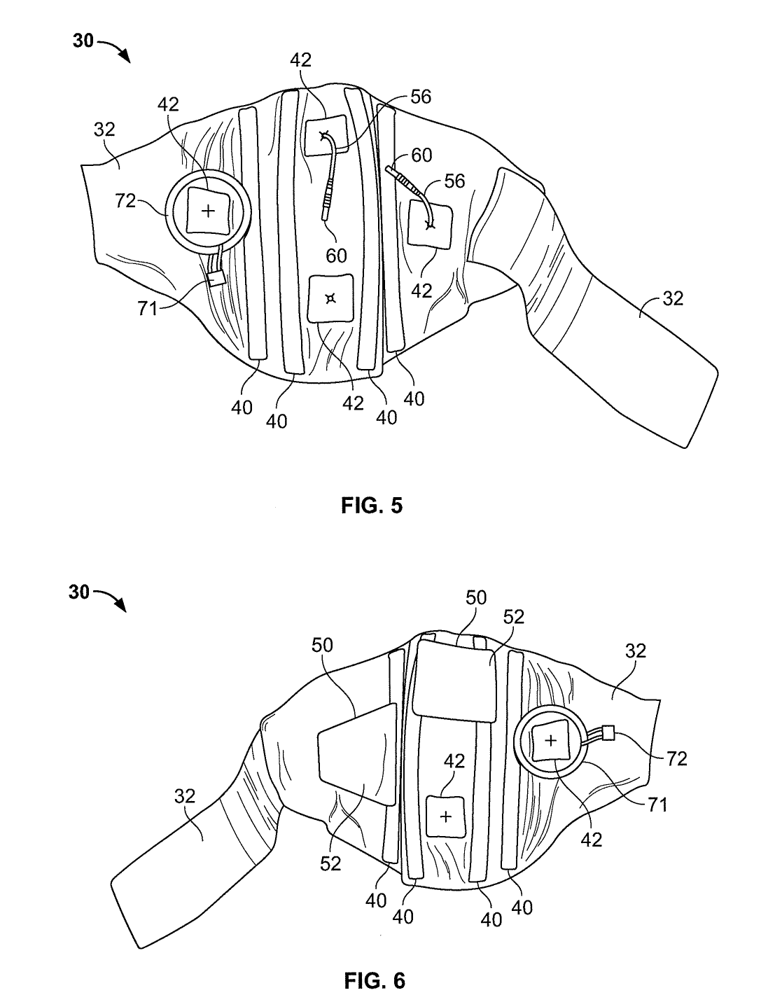

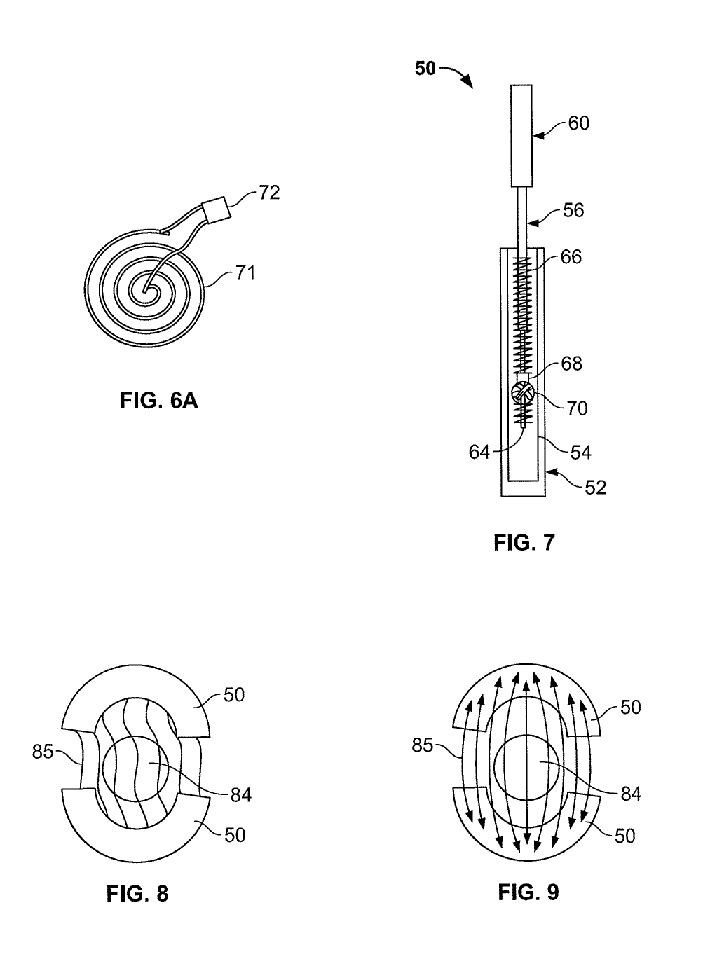

Regarding the time varying electromagnetic field (“TVEMF”) aspects of this invention wherein tissue regeneration is contemplated, the TVEMF generators are shown for purposes of illustration but not limitation. While they are shown on or in conjunction with only a few electrodes, it must be understood that they will be used in conjunction with any stimulation current where tissue regeneration is desired. In FIG. 1, the TVEMF generator 71 is shown as a coil wrapped around the arm and connected to a source 72 and focused on the tissue to be regenerated. In use, an electrical current is provided through the source and to the TVEMF generator for the repair and/or regeneration of tissue. The source of the electrical current that applies a TVEMF to the tissue can be programmed so that the TVEMF is applied to the tissue at the same time, alternating with, and/or sequentially with the stimulation current. In FIG. 1A, the TVEMF generator 71 is shown for illustration purposes as two flat sheets (it can also be one sheet) connected to a source 72. In FIG. 2, the TVEMF generator 71 is shown as a loop with the source 72 placed in conjunction with the programmable electrical stimulation device 20 so that the source controlling the electrical current of the TVEMF and the programmable electric stimulation device 20 that controls the stimulation current of the electrode 50 are controlled by the same unit. Because both currents, the electrical current supplying the TVEMF and the stimulation current supplying the stimulation through the electrode 50, may both be electrically driven, it is contemplated that in an embodiment, the electrical current and the stimulation current are one and the same depending on the configuration of the electrode and the TVEMF generator. In FIG. 3, the TVEMF generator 71 is shown as a coil with a source 72, and it extends around the entire body. In FIG. 4, the TVEMF generator 71 is shown as a helix connected to a source 72. Likewise, in FIG. 5 the TVEMF generator 71 is shown as a helix connected to a source 72. Another embodiment of the same helix form is shown in FIGS. 6 and 6A.

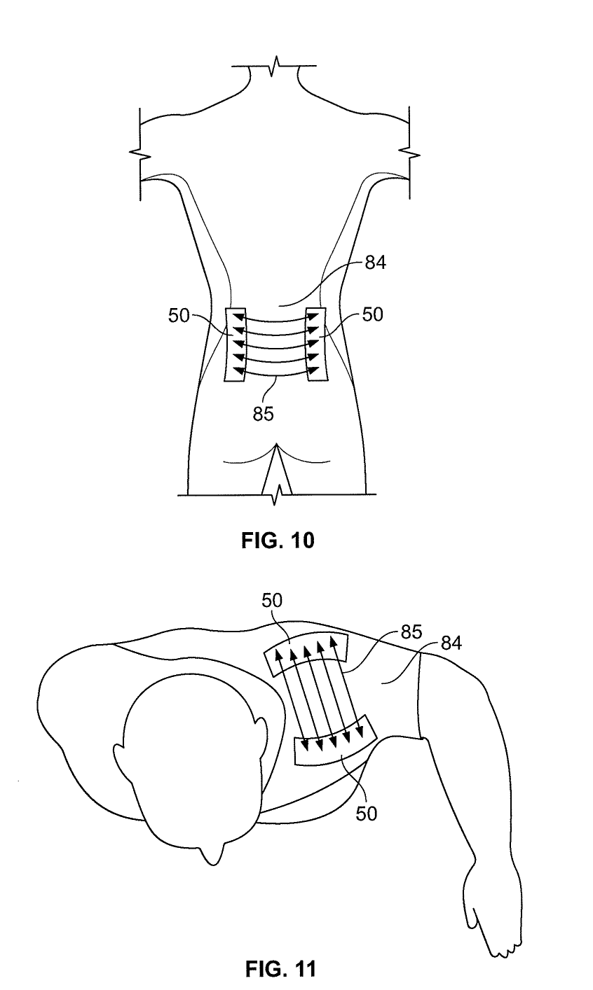

FIG. 8 shows an embodiment of the invention in use wherein the electrodes 50 direct a stimulation current 85 to prevent pain on the body area 84. In FIG. 9, still another embodiment of the stimulation current 85 applied to the body area 84 to prevent pain is shown. In FIG. 10, the electrodes 50 are shown on a person's back 84 with the stimulation current 85 traveling between the electrodes 50. In FIG. 11 the electrodes 50 with the stimulation current 85 are shown attached to a person's shoulder 84.

As various changes could be made in the system and method as it is contemplated in the present invention, without departing from the scope of the invention, it is intended that all matter contained herein be interpreted as illustrative and not limiting.

### DETAILED DESCRIPTION OF THE INVENTION

Therapeutic muscle stimulation, neuroprosthetic effects, and/or tissue regeneration may be achieved by using the present invention. The present invention may be useful for regenerating affected tissue while relieving pain, for instance, treating spinal cord injury, stroke, and other neurological conditions; and for the management of chronic pain. Therapeutic muscle stimulation may prevent or reverse muscle disuse atrophy, reduce spasticity, increase local blood flow, improve range of motion, and prevent deep thrombosis. When partial voluntary control remains, neuromuscular stimulation may increase the strength of the involved muscle groups. Neuroprosthetic effects may provide functional restoration by allowing a muscle or group of muscles to contract on command or automatically to produce a desired action such as opening a hand. The additional application of TVEMF in the system may regenerate tissue at the same time as the pain is relieved. The system may regenerate and relieve pain in various types of tissues including, but not limited to, neural tissue, muscle tissue, skin tissue, vascular tissue, adipose tissue, tissues and structures of the special sensory system, cartilage tissue, bone tissue, and interstitial tissue fluid.

To use the present invention, a person suffering from tissue damage, such as spinal cord injury or other neuromuscular trauma or other tissue damage or disorder, simply places an appropriately configured garment 30 on their body, connects the electrode 50 to the programmable electrical stimulation device 20, and can run a pre-programmed electrical stimulation routine. It is also contemplated that the wearer or user manually controls the programmable electrical stimulation device 20, thereby dispensing with the need to program the programmable electrical stimulation device 20, as preferred. In an embodiment, prior to the user placing wearable item 30 on their body and depending on the placement of the electrode 50 on or within the garment 30, an electrically conductive gel may be placed either directly on the bottom surface of first fabric layer 52 that contacts the skin of the user or within a closeable gel reservoir 38. Preferably, the electrically conductive gel is placed on a portion of the garment that is not water absorbent.

Concurrent with, alternating with, and/or sequentially with the pain-relieving stimulus, a TVEMF may be applied through the TVEMF generator so that tissue regeneration may be enhanced. The TVEMF generator and source of the system may be configured to produce a field amplitude less than 100 gauss peak-to-peak having a high slew rate with bipolar directional pulse in which the duty cycle is less than 1%.

This embodiment is but one example. Any number of different TVEMF signal embodiments are contemplated. For instance, another embodiment is envisioned to use a TVEMF generator configured to produce a relatively low peak magnetic field amplitude of less than 100 gauss having a slew rate greater than 1000 gauss per second driven by a bipolar square wave with a frequency less than 200 HZ and having duty cycle of less than 100%.

Yet another variation is to modify the latter embodiment by configuring the TVEMF generator to produce a slew rate greater than 1000 gauss per second for duration pulses of less than 1 ms. Still yet another variation is to modify the latter embodiment by configuring the TVEMF generator to produce magnetic slew rate bipolar pulses with superimposed square waves to generate complex linear summations of magnetic waveforms.

Preferably, the TVEMF generated field should exhibit relatively sharp breaks when graphed, such as those of square waves or delta wave. The TVEMF field can be applied using any TVEMF generator known in the art including, but not limited to, a helix, a coil, a solenoid, at least one loop, an antennae, a Helmholtz coil configuration, and an electrode. The TVEMF generator can also be configured as one or two sheets or one or two blankets connected to a source to cause the TVEMF to penetrate the tissue to be regenerated. One preferred embodiment is that the TVEMF field exhibits a relatively nearly uniform magnetic field strength throughout the targeted body area. Alternatively, another embodiment is that the TVEMF field exhibits a relatively nonuniform magnetic field strength throughout the targeted body area.

The TVEMF field can also be applied utilizing a flux concentrator to provide spatial gradients of magnetic flux and magnetic flux focusing within body tissue to be encouraged to be regenerated and/or repaired. Other embodiments include configuring the TVEMF generator to produce time varying electromagnetic field fluxes that can apply static or dynamic magnetic field gradients to the targeted body part, in which the electrical stimulation and time varying electromagnetic field can be applied to the tissue either at the same time or applied to the tissue at different times but applied sufficiently close to each other to regenerate the tissue and to reduce pain.

In another embodiment, such as rotator cuff repair, it may be necessary or desirable to infuse or introduce the damaged area with/to an abundance of stem cells (preferably expanded adult stem cells) to speed repair. In such cases, the cells may be embedded in a material to keep them in place during the repair. This can be accomplished by embedding the stem cells in a biogel, such as, but not limited to: hydrogel polymers, polymerized polyethylene glycol diacrylate, polylactic acid, polyglycolic acid, polymerized polyethylene glycol dimethylacrylate and mixtures thereof. Alternatively, the cells may be introduced to the targeted tissue area by delivering cells to a tissue such as through a syringe or other ways of cell delivery to tissues well known in the art. Although any number of adult stem cells can be used in this invention, a preferred range of cells may be from about 4×10⁴ to about 6×10⁷. The number of cells that are therapeutic will depend on the extent of repair and/or regeneration of tissue necessary and the method of delivering the cells to the tissue area. There is no upper limit to the number of adult stem cells that can be introduced to the affected tissue. Not to be bound by theory, but it is thought that the body's natural system disposes of adult stem cells that are not useful, in excess, or unnecessary for the repair. Some examples of adult stem cells that can be used include, but are not limited to, mesenchymal cells, CD34+ cells, CD 133+ cells, non-terminally differentiated cells, and progenitor cells. The cells can be from the same person as the user of the system wherein they are autologous, or from a different person(s) wherein they are considered allogeneic. Moreover, a pool of stem cells can be used from different sources including, but not limited to, a blood bank, a storage facility, donor(s), and from a combination of the same.

A further understanding of this invention can be obtained by reference to certain specific examples detailed below, which are provided for purposes of illustration only and are not intended to be all inclusive or limiting.

### EXAMPLES

An exemplary embodiment of the system and device of the present invention is the shorts stimulator combination (see FIGS. 3-4). This embodiment includes a lycra-spandex, electrode-embedded garment and multi-channel micro-processor muscle stimulator that is worn on the body of a spinal cord injured patient throughout the day or for short durations such as 1-2 hours. This embodiment enables the user to select from a variety of programmable electric stimulation device modes and parameters including menu options that deliver exercise options, standing, weight-shift and other muscle functionality. The TVEMF generator regenerates the injured area concurrently with the stimulation. The shorts can be worn for extended hours without a degradation of the contact between skin and electrode enabling patients to wear the device for many hours without having to reapply gel. The programmable electric stimulation device provides modes and parameters from which to choose targeted to improving sitting posture in the spinal cord; correcting spinal alignment problems; reciprocal stimulation to the gluteals and hamstrings left to right after seating pressures; reducing pressure; improving blood flow to the gluteal muscles and otherwise preventing skin breakdown in patients confined to wheelchairs; standing at will; controlling pain; improving circulation; and providing exercise.

Another exemplary embodiment of the system and device of this invention is the belt (see FIGS. 5-6) which stimulates abdominal muscles. This embodiment provides an additional electrode belt that conforms to the pendulous shape of the typical quadriplegic or paraplegic having paralyzed abdominal muscles. The belt is designed to maximize stretch of the fabric that comprises the front panel of the belt. Flexible, water-resistant and insulating material, for instance Darlexx, allows the fabric to stretch in two dimensions to accommodate any convexity of the exterior wall. Tapering the front panel at specific locations on the belt creates an initial convexity and the use of detachable electrodes permits the front panel to stretch as needed. Only a small section of the electrode 50, typically the center, is fixed to the front panel, either by Velcro, stitching, or other means of attachment or association with the fabric of the garment known in the art.

In each instance where tissue stimulation is used, a corresponding and suitable TVEMF generator is used to regenerate the applicable tissue. It should be understood that the electrical pain relief stimulation and TVEMF generation can be provided from separate units or from the same unit programmed to provide the output in alternating fashion or at the same time. The source for the TVEMF and the programmable electric stimulation device can be combined into one unit or can preferably be separate, are programmable, and can be configured to remotely communicate with each other. The system of this invention is durable and easy to place on the body and use, and has minimal user discomfort, if any, which is more than overcome by the pain relief and tissue regeneration that occurs.

While the present invention has been illustrated by the description of exemplary embodiments thereof, and while the embodiments have been described in certain detail, it is not the intention of the Applicant to restrict or in any way limit the scope of the appended claims to such detail. Additional advantages and modifications will readily appear to those skilled in the art.

Therefore, while embodiments have been herein described, those skilled in the art will understand the present invention to include various changes and modifications. The scope of the invention is not intended to be limited to the above-described embodiments, and departures may be made from such details without departing from the spirit or scope of the general inventive concept.

We claim:

1. A system for transcutaneous tissue stimulation and regeneration comprising:
a. a garment comprising a fabric of non-conductive material;
b. at least one electrode associated with the fabric of the garment;
c. a programmable electrical stimulation device operatively connected to the at least one electrode such that the electrode provides a stimulating current;
d. a time varying electromagnetic field generator; and
e. a source for supplying the time varying electromagnetic field generator with electrical current to cause it to generate a time varying electromagnetic field.

2. The system of claim 1 wherein the electrode is removably attached to the fabric.

3. The system of claim 1 wherein the electrode is embedded within the fabric.

4. The system of claim 1 wherein the garment comprises a vest, briefs, belt, shorts, brace, sling, immobilizer, and combinations thereof.

5. The system as in claim 1 wherein the electrode comprises:
a. a first fabric layer;
b. at least one piece of conductive material in contact with the first fabric layer;
c. a length of electrical wire wherein a portion of the electrical wire is un-insulated, and wherein the un-insulated portion of the wire is in contact with the electrode; and
d. a second fabric layer of conductive material connected to the un-insulated portion of the electrical wire.

6. The system of claim 5 wherein the conductive material contains embedded silver.

7. The system of claim 1 wherein the electrode is covered at least in part with a conductive gel.

8. The system of claim 1 wherein the time varying electromagnetic field has a peak field amplitude less than 100 gauss having a slew rate greater than 1000 gauss per second driven by a bipolar square wave with a frequency of less than 200 Hz and having a duty cycle of less than 100%.

9. The system of claim 1 wherein the time varying electromagnetic field has a slew rate greater than 1000 gauss per second for duration pulses of less than 1 ms.

10. The system of claim 1 wherein the time varying electromagnetic field is applied using a coil to create a nearly uniform field strength throughout the targeted body tissue.

11. The system of claim 1 wherein the time varying electromagnetic field is not spatially uniform.

12. The system of claim 1 wherein the time varying electromagnetic field is applied utilizing a flux concentrator to provide spatial gradients of magnetic flux and magnetic flux focusing within the body tissue to be generated.

13. The system of claim 1 further comprising connectors, power source, wires, electrodes, and at least one attachment device and wherein the programmable electrical stimulation device, connectors, power source, wires, electrodes, and at least one attachment device are modular.

14. The system of claim 1 wherein the programmable electrical stimulation device has modes and parameters and wherein the modes and parameters are controlled by at least one from the group consisting of remotely, interface with a computer system, the internet, a telecommunication device, and automated feedback control involving biofeedback signals.

15. The system of claim 1 wherein the programmable electrical stimulation device has modes and parameters and wherein the modes and parameters are selected from among a pre-set number of modes.

16. The system of claim 1 wherein the programmable electrical stimulation device has modes and parameters and wherein the modes and parameters are smoothly adjustable by the user.

17. The system of claim 1 wherein the programmable electrical stimulation device has modes and parameters and wherein the modes and parameters are not adjustable by the user.

18. The system of claim 1 wherein the programmable electrical stimulation device has modes and parameters and wherein the modes and parameters adjust or vary over time according to a prescribed protocol.

19. The system of claim 1 wherein the programmable electrical stimulation device is operatively connected to sensors for stimulation current, magnetic flux, temperature or impedance.

20. The system of claim 1 wherein the programmable electrical stimulation device is configured to drive more than one set of electrodes, which may be located at more than one location on the body, or on more than one garment, and may be operated independently or in synchronized fashion.

21. The system of claim 1 further comprising at least one adult stem cell.

22. The system of claim 1 wherein the programmable electrical stimulation device and the source are one unit.

23. A method of simultaneously regenerating tissue and reducing pain, said method comprising:
subjecting the tissue to a stimulation current and a time varying electromagnetic field simultaneously or in a coordinated fashion to reduce pain and regenerate the tissue.

24. The method of claim 23 wherein the tissue to be stimulated is selected from the group consisting of neural tissue, muscle tissue, skin tissue, vascular tissue, adipose tissue, tissues and structures of the special sensory system, cartilage tissue, bone tissue, implanted material, and interstitial tissue fluid.

25. The system of claim 24 wherein the implanted material is a biogel.

26. The system as in claim 25 wherein the biogel is selected from the group comprising hydrogel polymers, polymerized polyethylene glycol diacrylate, polylactic acid, polyglycolic acid, polymerized polyethylene glycol dimethylacrylate and mixtures thereof.

27. The method of claim 23 wherein the time varying electromagnetic field has a magnetic field amplitude less than 100 gauss peak-to-peak having a slew rate with bipolar directional pulse in which the duty cycle is less than 1%.

28. The method of claim 23 wherein the time varying electromagnetic field is applied using a coil to create a nearly uniform field strength throughout the targeted body area.

29. The method of claim 23 wherein the time varying electromagnetic field is not spatially uniform.

30. The method of claim 23 wherein the time varying electromagnetic field is applied utilizing a flux concentrator to provide spatial gradients of magnetic flux and magnetic flux focusing within the body tissue to be regenerated.

31. The method of claim 23 wherein the time varying electromagnetic field flux is configured to apply static or dynamic field gradients to the targeted body part.

32. The method of claim 23 further comprising the step of introducing at least one stem cell to the tissue.

33. A method for regenerating tissue and minimizing pain comprising the step of:
wearing a system comprising a garment having a non-conductive fabric; at least one electrode attachable to or embedded within the fabric of the garment; a programmable electrical stimulation device with modes and parameters and operatively connected to at least one electrode; a time varying electromagnetic field generator associated with the fabric of the garment; and a source having modes and parameters to supply the time varying electromagnetic field generator with electrical current to cause it to generate a time varying electromagnetic field;
stimulating the tissue with a stimulation current to the tissue to minimize the pain;
applying a time varying electromagnetic field focused on the tissue to regenerate the tissue;
controlling the modes and parameters of the source; and
controlling the modes and parameters of the programmable electrical stimulation device.

34. The method as in claim 33 wherein the electrode comprises:
a. a first fabric layer;
b. at least one piece of conductive material in contact with the first fabric layer;
c. a length of electrical wire wherein a portion of the electrical wire is un-insulated, and wherein the un-insulated portion of the wire is in contact with the electrode; and
d. a second fabric layer of conductive material connected to the un-insulated portion of the electrical wire.

35. The method of claim 33 wherein the electrode is covered at least in part with a conductive gel.

36. The method of claim 33 wherein the time varying electromagnetic field has a field amplitude less than 100 gauss having a slew rate greater than 1000 gauss per second driven by a bipolar square wave with a frequency of less than 200 Hz and having a duty cycle of less than 100%.

37. The method of claim 33 wherein the time varying electromagnetic field has a slew rate greater than 1000 gauss per second for duration pulses less than 1 ms.

38. The method of claim 33 wherein the time varying electromagnetic field is applied using a coil to create a substantially uniform field strength throughout the targeted body area.

39. The method of claim 33 wherein the time varying electromagnetic field is not spatially uniform.

40. The method of claim 33 wherein the time varying electromagnetic field is applied utilizing a flux concentrator to provide spatial gradients of magnetic flux and magnetic flux focusing within the tissue to be regenerated.

41. The method of claim 33 wherein the time varying electromagnetic field flux is positioned as to apply static or dynamic field gradients to the targeted body part.

42. The method of claim 33 wherein the programmable electrical stimulation device utilizes micro-power design techniques.

43. The method of claim 33 wherein the system further comprises connectors, power source, wires, electrodes, and attachment means and wherein the programmable electrical stimulation device, connectors, power source, wires, electrodes, and attachment means are modular.

44. The method of claim 33 wherein the step of controlling the programmable electrical stimulation device modes and parameters is selected from the group consisting of remote wireless communication, through interface with a computer system, the Internet, a telecommunications device, and a combination thereof.

45. The method of claim 33 wherein the controlling steps are determined by automated feedback control involving biofeedback signals.

46. The method of claim 33 wherein the controlling steps allow adjustment of programmable electrical stimulation device modes and parameters related to the group consisting of electrical current, magnetic flux, temperature, and impedance.

47. The method of claim 33 wherein the stimulation current and time varying electromagnetic field are provided to the tissue alone, simultaneously, in a synchronized alternating pattern, or a combination thereof.

48. The method of claim 33 further comprising the step of introducing at least one adult stem cell to the tissue to be regenerated.
